(19) United States
(12) Patent Application Publication
(10) Pub. No.: US 2010/0137689 A1
(43) Pub. Date: Jun. 3, 2010

(54) LAMINAR FLOW ENDOSCOPE

(76) Inventor: James K. Brannon, Leawood, KS (US)

Correspondence Address: INTELLECTUAL PROPERTY CENTER, LLC, 9233 WARD PARKWAY, SUITE 100, KANSAS CITY, MO, 64114, US

(21) Appl. No.: 12/706,706

(22) Filed: Feb. 16, 2010

Related U.S. Application Data
(63) Continuation-in-part of application No. 10/928,553, filed on Aug. 26, 2004, now Pat. No. 7,445,595.
Application No. 10/928,553 is a division of application No. 10/957,817, filed on Oct. 4, 2004, now abandoned.
(60) Provisional application No. 61/152,925, filed on Feb. 16, 2009.

(51) Int. Cl. A61B 1/12

(52) U.S. Cl. 600/156

(57) ABSTRACT

The present invention is directed to a laminar flow endoscopic instrument for laminar transmission of irrigation fluid between an incision towards a zone of visualization associated with a surgical site, the endoscopic portal comprising an osteoendoscopic cylinder having an outer contact surface encircling an inner visual surface and extending between a proximal handle end and a distal endoscopic end with a directional surface extending therefrom and providing laminar flow of irrigation fluid.

### CROSS REFERENCE TO RELATED APPLICATION

This application is a non-provisional application claiming the benefit of the earlier filed provisional application No. 61/152,925 filed on Feb. 16, 2009 which is a continuation in part of U.S. patent application Ser. No. 10/928,553 filed on Aug. 24, 2004 which is a divisional of U.S. patent application Ser. No. 10/957,817, filed Sep. 19, 2001, the contents of which are hereby incorporated by reference.

### FIELD OF THE INVENTION

This invention relates generally to endoscopic surgical instruments. Specifically, the present invention relates to a laminar flow endoscopic trephine which provides visual clarity during surgical procedures at a surgical site that can receive plural surgical instruments and a method for using the same in orthopedic procedures.

### BACKGROUND OF THE INVENTION

### 1. Field of the Invention

Medical instruments are often utilized in treatment of various disorders and medical conditions. Often these medical instruments are introduced through the use of an endoscope.

Endoscopy is generally used to effect removal of the unwanted or damaged tissues from a patient in a manner that is less invasive than completely opening up the tissue and using traditional tools. However, use of traditional endoscopy techniques can cause increased pressure within the surgical site which limits the visibility of any ongoing vascular activity and can cause further injury to the patient resulting in increased patient recovery and additional costs based upon the increased pre-operative and post-operative hospital stays and the need to repair, replace or correct injuries.

Generally, prior endoscopes allow a doctor to insert a medical instrument through the endoscope to the surgical site through an incision to observe, diagnosis or treat the patient, however, based upon the surrounding debris and visual obstructions, these procedures often took longer than necessary and resulted in the unnecessary removal of tissue, which obstructs the view of the surgeon during the procedure. In addition, blood and tissue can collect along the surgical site forming an obstruction along the front of the endoscope, which are non-transparent and limit the visual control of the endoscope or use of the instruments.

During the endoscopic procedures it is important to maintain the visibility of the surgical site from surrounding fluids, surgical debris and contaminants. Some current attempts at treating or examination of medical conditions involve the use of endoscopes connected to vacuum sources or irrigation sources to inject or remove fluids from the surgical site. However, many of these sources cause additional obstructions within a surgical site limiting the visibility of the surrounding areas.

Additionally, some endoscopes provide an irregular surface associated with the injection of fluid which causes turbulent flow around the surgical site, damaging surrounding tissue and agitating surrounding debris, again decreasing visual clarity. Some attempts to improve the visual clarity include spacing the endoscope a distance from the surgical site to create a non-flow region to limit the turbulence, however, these attempts limit the ability to treat the surgical site and limit the ability to clear the visual field during surgery. Additionally, by spacing the endoscope a distance from the surgical site, gas bubbles may be formed in the non-flow regions associated with the surgical site during some surgical procedures. These gas bubbles can collect at the surgical site, again limiting the visual clarity during surgery. Therefore, it would be beneficial to provide an endoscope which improved the visual clarity of the surgical site during surgery.

Medical instruments used for examining or treating a patient are also utilized by the endoscope and may also cause various debris or obstructions, surrounding the desired surgical site which decrease visibility and increasing the potential injury to the patient. Some attempts to remove the obstructions include the use of additional instruments to manipulate the debris or obstructions such as cutting tools, mechanical jaws or electrocautery devices. Often the use of these instruments tears, rips or injures the patient or creates additional obstructions or debris which further clouds the visible clarity of the surgical site and increases the risk of patient injury.

It therefore would be beneficial to provide an improved endoscopic trephine as further described below which addresses some of the concerns addressed above.

### SUMMARY OF THE INVENTION

According to the present invention, a laminar flow endoscopic instrument is provided for laminar transmission of an irrigation fluid between an incision towards a zone of visualization associated with a surgical site, said endoscopic portal comprising an osteoendoscopic cylinder having an outer contact surface encircling an inner visual surface and extending between a proximal handle end and a distal endoscopic end; a handle extending from the proximal handle end and presenting the osteoendoscopic cylinder for coaxial receipt of a surgical instrument along said inner visual surface and a directional guide associated with said distal endoscopic end and having a directional surface providing laminar flow of irrigation fluid longitudinally towards the surgical site.

### BRIEF DESCRIPTION OF THE DRAWINGS

FIG. 1 is a perspective view of the laminar flow endoscope associated with a femoral head.

FIG. 2 is a right side perspective view of the laminar flow endoscope according to FIG. 1.

FIG. 3 is a left side bottom perspective view of the laminar flow endoscope according to FIG. 1.

FIG. 4 is a right side bottom perspective view of the laminar flow endoscope according to FIG. 1.

FIG. 5 is a right side cross-section of the laminar flow endoscope according to FIG. 1.

FIG. 6 is a right side elevation of the laminar flow endoscope according to FIG. 1.

### DETAILED DESCRIPTION OF THE INVENTION

### I. Introduction.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

### II. Laminar Flow Endoscope.

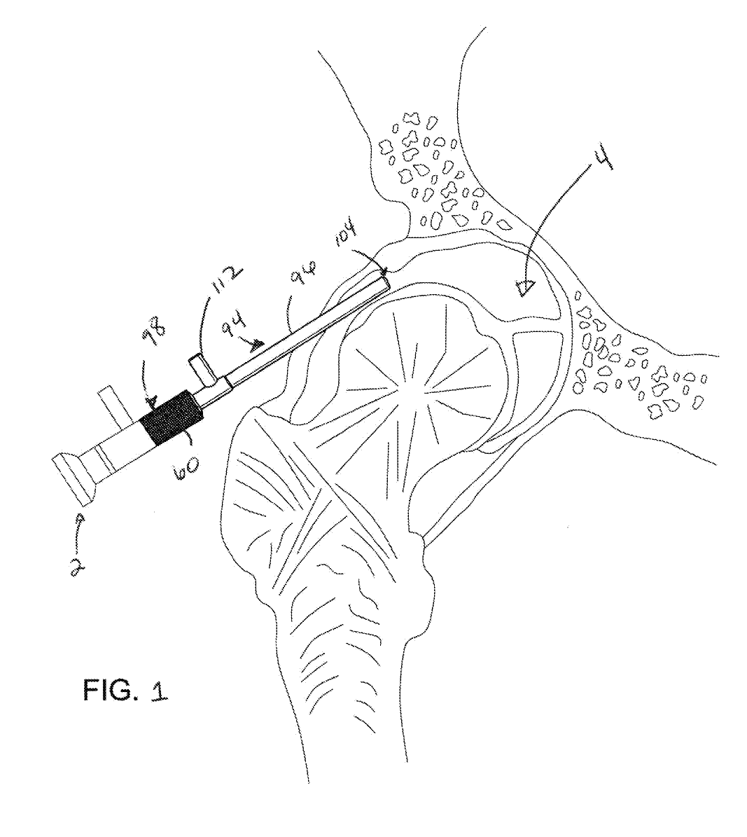

FIG. 1 depicts a femoral head 4 in receipt of a laminar flow endoscope generally referred to herein by reference numeral 10. As illustrated the laminar flow endoscope 10 generally extends cylindrically between a proximal handle end 98 associated with a handle 60 towards a distal endoscopic end 104, and including an inner visual surface 106 associated with a centrally disposed lumen 20 and an outer contact surface 96 circumscribing the inner visual surface 106. The endoscope 10 is generally is made from a rigid material such as stainless steel and is adapted for the receiving surgical instruments such as, but not limited to the laparoscope 2 illustrated in FIG. 1. Generally, the endoscope 10 facilitates extending various surgical instruments towards a surgical site which is illustrated in FIG. 1 near the femoral head 4. Optionally, the laminar flow endoscope 10 is illustrated with a side opening 112 oriented normal to an osteoendoscopic cylinder 94 generally defined by the outer contact surface 96 and the inner visual surface 106.

The osteoendoscopic cylinder 94 according to the present invention includes an elongated sleeve 12, which, for example, can take the form of a cannula or portal sleeve or other structure providing a passage through a cavity wall, extends from an open proximal end 99 associated with the proximal handle end 98 towards the distal endoscopic end 104 adapted for being disposed at an internal surgical site in a body cavity, the proximal handle end 98 adapted for being disposed externally from the cavity, the lumen 20 extending therebetween. The open proximal end 99 of sleeve 12 is illustrated as being generally integral with the endoscope 10, however, other known configurations may be alternatively utilized.

The outer contact surface 96 being generally of a dimension adapted for contact with a longitudinal canal surface so as to tamponade bleeding therefrom. The outer contact surface 96 is further of a size and dimension adapted to establish a hermetic seal at the juncture thereof and the longitudinal canal surface 102. The osteoendoscopic cylinder 94 is of a size and dimension adapted to receive an endoscope coaxially along the inner visual surface 106 and further includes the proximal handle end 98 and is shown having been engaged by the handle 60. In FIG. 1, the distal endoscopic end 104 is shown in proximity to a segment of osteonecrotic bone. Operationally situated about the proximal handle end 98 is the side opening 112 serves to supply an irrigation fluid through the osteoendoscopic cylinder 94 to the distal endoscopic end into the surgical site, where a flow-influencing means is provided to ensure that the irrigation fluid reaches a zone of visualization associated with the surgical site. The side opening 112 is preferably adapted of a size and shape to receive and transmit the irrigation fluid.

Additionally, the side opening 112 may be adapted of sufficient size and shape to induce a negative pressure upon the zone of visualization and may include a vacuum and a transparent tube adapted for evacuating a quantity of osteonecrotic bone fragments debrided from the femoral head 4. The negative pressure provides a low pressure environment within the femoral head 4 so as to decompress an elevated intraosseous pressure therein. Further, the negative pressure may also induces blood to flow from the cancellous bone.

The distal endoscopic end 104 positioned within the femoral head 4, is illustrated in FIG. 1 situated juxtainferior to the segment of osteonecrotic bone. A laparoscope 2 is shown having been advanced into the osteoendoscopic cylinder 94 in FIG. 1, substantially coaxially along the inner visual surface 106 for operating on the surgical site. The laparoscope 2 passes into the osteoendoscopic cylinder 94 after first passing through the handle 60 and over a proximal stabilizing support 114.

The proximal stabilizing support 114, being of a size and dimension to allow distal and proximal advancement of the laproscope 2 or other surgical instruments within the osteoendoscopic cylinder 94 while supporting the received instrument and maintaining visualization of the surgical site about the zone of visualization. As illustrated the proximal stabilizing support may be circularly, or otherwise, shaped to present a seal and support the received surgical instrument along an axis extending along the inner visual surface. The proximal stabilizing support 114 is further of a size and dimension adapted to present a seal, limiting the flow of air at the juncture thereof and the longitudinal surface of the laproscope 2. With the laproscope 2 received by the osteoendoscopic cylinder 94, the surgeon may manipulate the optics thereof so as to visually observe the surgical site at the zone of visualization associated therewith.

The handle 60 has a pair of internally positioned fastening members 60a, 60b for releasable engagement with an internally received instrument like the laparoscope 2. The fastening members 60a, 60b are generally positioned within a handle end 98 opposite the elongated sleeve 12 and having an opening 21 therein communicating with lumen 20. The opening 21 in handle end 98 is axially aligned with lumen 20 and has a size corresponding to the cross sectional size of an instrument to be introduced through the endoscope for surgical use. Handle 60 also includes a cylindrical shaft 54 extending axially from the handle end 98 to a lateral port of entry 50. The handle 60 is generally of uniform thickness and has a frictional surface along the circumference thereof to facilitate grasping.

When an instrument having a cross sectional size corresponding to the handle end 98 is received within the handle 60, the instrument may be rotated for engagement by the fastening members 60a, 60b. In addition to the handle end 98, the lumen 20 may also be configured with a specific cross section to receive instruments of various cross sectional sizes. In this way, plural endoscopes may be used for varying dimensioned instruments or alternatively, an enlarged endoscope with a larger handle end 60 and lumen 20 may be used to facilitate varying dimensioned instruments.

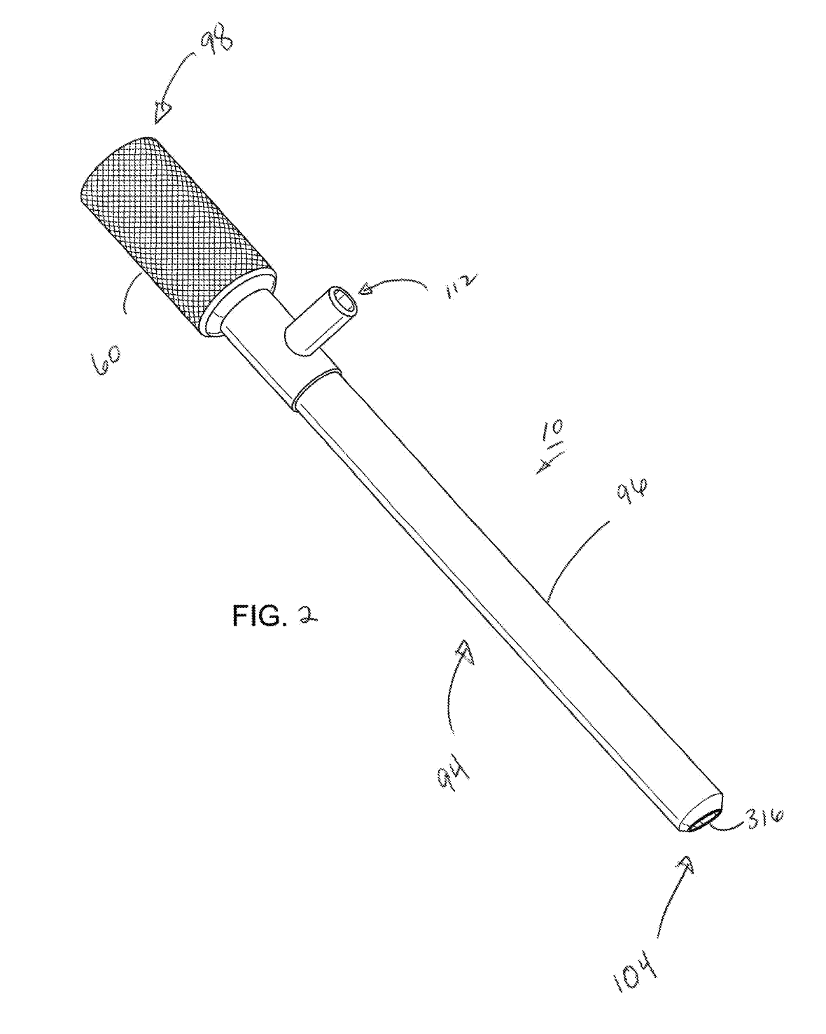

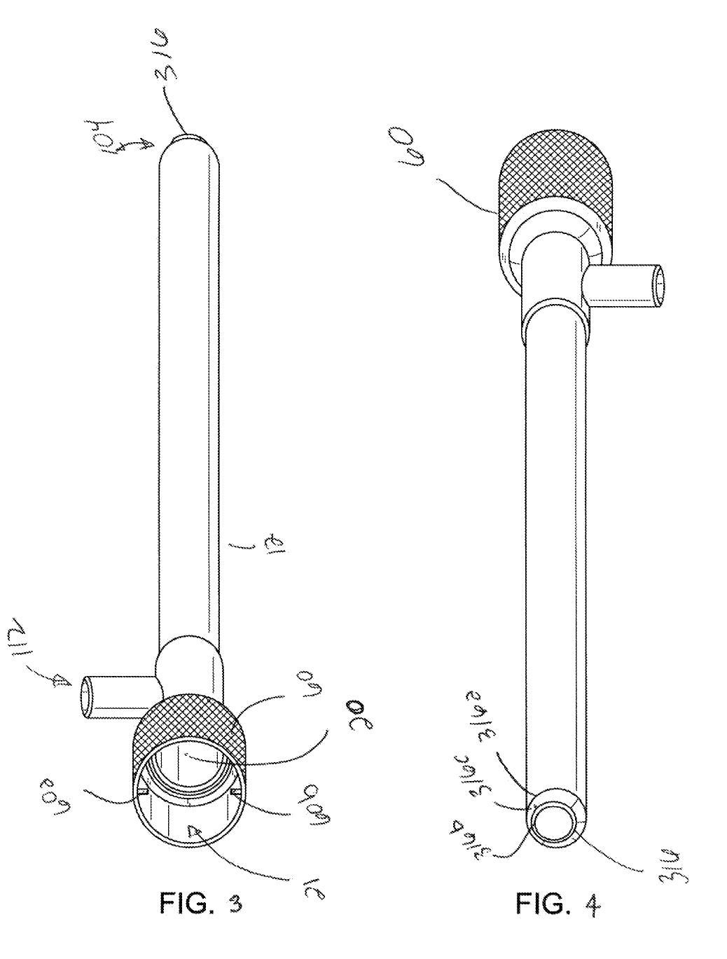

In addition, a directional guide 316 is illustrated in FIGS. 2-4, associated with the distal endoscopic end 104 and extending from the sleeve 12. Generally, the directional guide 316 is of a size and shape adapted to influence the visibility of the zone of visualization associated with the surgical site. Generally, directional guide 316 delivers the irrigation fluid to the surgical site while promoting laminar flow of the irrigation fluid.

Specifically, the directional guide 316 in combination with the inner visual surface 106 reduces the turbulence of the flow, thereby promoting laminar flow of the transmitted irrigation fluid to control and direct the dispersed fluid for effecting surrounding debris associated with the surgical site and providing unobstructed visual control of the surgical site. In the illustrated embodiment of FIG. 4, the directional guide 316 includes a directional surface comprised of an outer ring 316a and an inner ring 316b separated by an arcuate surface 316c, the outer ring 316a associated with the distal end 104 of the osteoendoscopic cylinder 94 and the inner ring 316b adapted for placement near the surgical site. During the transmission of media through the osteoendoscopic cylinder 94 to the surgical site, the directional guide 316 transmits the media, such as surgical irrigation fluid, longitudinally along a laminar axis preferably aligned with the osteoendoscopic cylinder 94. By transmitting the media along the laminar axis, the osteoendoscopic cylinder 94 promotes laminar flow of the dispersed media, thereby desirably influencing the surrounding debris to improve the surgical site. For example, the laminar axis may be aligned with surrounding obstructions and the media may be transmitted over the directional guide and towards the surrounding obstruction, presenting the media at the desired location in a shaped laminar pattern. The shape of the directional guide 316 in combination with the inner visual surface 106 presents the shaped laminar pattern and as desired reduces the turbulence of the media flow, thereby promoting laminar flow and resulting in improved visual clarity at the surgical site.

The irrigation fluid travels along the inner visual surface 106 which delivers the irrigation fluid to the surgical site at the distal endoscopic end 104. The irrigation fluid is introduced into the osteoendoscopic cylinder 94 at the proximal handle end 98 through an irrigation connector (not shown) for connecting an irrigation tube (also not shown). The irrigation fluid is transmitted axially along the inner visual surface 106 to the distal endoscopic end 104 where it is dispensed to the surgical site. While we have generally referred to the irrigation fluid as being a fluid, it may be any type of media to be dispensed to the surgical site for improving the visual clarity at the zone of visualization through laminar flow.

The distal endoscopic end 104 of the endoscope 10 is disposed generally perpendicularly to the direction of flow of the irrigation fluid, the directional guide 316 providing the necessary bias to promote laminar flow thereat. Alternatively, the directional guide 316 may be complementary inclined to mate with received surgical instruments extending through the endoscope 10 via a shaft connectably secured to the instrument. The directional guide 316 thereby being adapted to promote laminar flow through the inner visual surface 106 along the shaft of the received instrument. In addition, a suction connector may be utilized to remove any dispersed media away from the surgical site.

During operation, the surgeon may position the side opening 112 in a posterior direction, thereby directing the dispensed media towards a target associated with the surgical site, the target presenting a visual obstruction. In this regard, the visual quality of the surgical procedure may be improved.

An optional multiple branch passageway in communication with the handle end 98 and the side opening 112 may be utilized in the present invention to facilitate plural instruments or procedures simultaneously. As illustrated in FIG. 2, the multiple branch passageway, in the nature of a “T”, is illustrated with the side opening 112 extending outwardly from the central passageway which stretches from the lateral port of entry 50 associated with the handle 60 towards the distal endoscope end 106. The multiple branch passageway may further include another branch for an additional instrument, procedure or treatment as desired. Additionally, a flow regulator 23 may be utilized at an opening to the osteoendoscopic cylinder 94, spaced apart from the distal endoscopic end a distance to maintain laminar flow while providing desirable flow characteristics to any transmitted media along the osteoendoscopic cylinder 94.

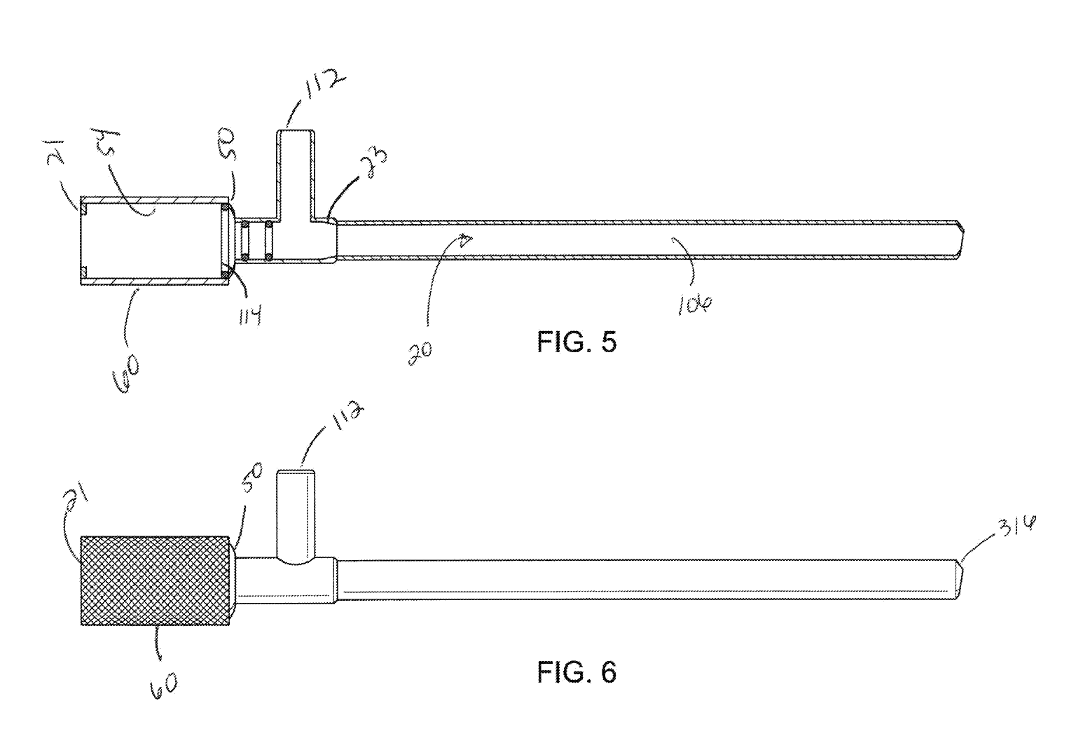

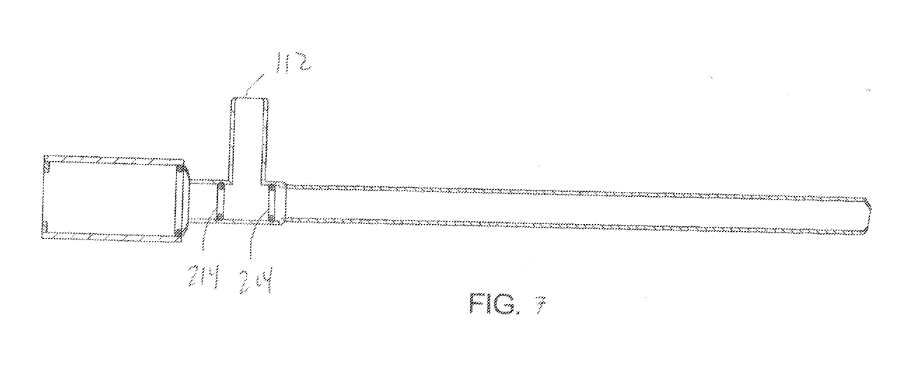

A proximal stabilizing support 114 is illustrated in FIG. 5 adapted to provide hermetic seal at the juncture of the passageway and the received surgical instruments at the lateral portal of entry 50. In addition, additional lateral supports may be provided within the passageway for providing operational benefits including supporting the received instruments.

An alternative embodiment including a proximal stabilizing support pair 214 is illustrated in FIG. 7 adapted to provide a hermetic seal near the side opening 112 with one of stabilizing support pair being positioned between the proximal handle end 98 and the side opening 112 and the other one of said stabilizing support pair being positioned between said osteoendoscopic cylinder 94 and the side opening 112. In addition, the stabilizing support pair 214 may be utilized to provide additional operational benefits including annular support of the received instruments. The radius of the proximal stabilizing support pairs may be further configured, providing a non-hermetic seal at the juncture of the side opening and the elongated sleeve 12. Transmitting the fluid from the side opening 112 along the elongated sleeve 12 towards the distal endoscopic end 104 may further support the laminar fluid flow.

Optionally, the endoscope may be utilized with an endoscopic portal having an elongated sleeve presenting a central channel for receiving the endoscope between an anterior surface and a posterior surface. In use, the endoscopic portal provides repeated entrance to a guided passageway between the incision to the surgical site.

While the invention has been described with respect to specific examples including presently preferred modes of carrying out the invention, those skilled in the art will appreciate that there are numerous variations and permutations of the above described systems and techniques that fall within the spirit and scope of the invention as set forth in any claims.

Having thus described the invention, what is claimed as new and desired to be secured by Letters Patent is as follows:

1. An endoscopic portal for laminar transmission of an irrigation fluid from an incision towards a zone of visualization associated with a surgical site, said endoscopic portal comprising:
an osteoendoscopic cylinder having an outer contact surface encircling an inner visual surface and extending between a proximal handle end and a distal endoscopic end;
a handle extending from the proximal handle end and presenting the osteoendoscopic cylinder for coaxial receipt of a surgical instrument along said inner visual surface; and
a directional guide associated with said distal endoscopic end having a directional surface providing laminar flow of irrigation fluid longitudinally towards the surgical site.

2. The device according to claim 1, further comprising a proximal stabilizing support dimensioned to slidably receive a surgical instrument while restricting fluid flow at the juncture of said proximal stabilizing support and said surgical instrument, whereby the surgical instrument can be manipulated to the surgical site.

3. The device according to claim 2, wherein said proximal stabilizing support is located between the osteondoscopic cylinder and the handle.

4. The device according to claim 1 wherein said osteoendoscopic cylinder further comprises a side opening associated with said proximal handle end, said side opening in fluid communication with said distal endoscopic end.

5. The device according to claim 5, further comprising a proximal stabilizing support pair dimensioned to slidably receive a surgical instrument while restricting fluid flow at the juncture of said proximal stabilizing support pair and said surgical instrument, whereby one of said proximal stabilizing support pair is located between the osteondoscopic cylinder and said side opening and the other one of said proximal stabilizing support pair is located between said proximal handle end and said side opening.

6. The device according to claim 1, further comprising a multiple branch passageway associated with said proximal handle end and including a plurality of openings angularly converging towards a central passageway.

7. The device according to claim 1, further comprising at least one fastening member for releasable engagement of at least one surgical instrument.

8. The device according to claim 1, wherein said directional guide is comprised of an outer ring and an inner ring separated by an arcuate surface.

9. An endoscopic portal extending between an incision and a surgical site, said endoscopic portal comprising:
an osteoendoscopic cylinder having an outer contact surface encircling an inner visual surface and extending between a proximal handle end and a distal endoscopic end;
a handle extending from the proximal handle end and presenting the osteoendoscopic cylinder for coaxially receipt of a laparoscope along the inner visual surface;
a proximal stabilizing support dimensioned to slidably support said laparoscope received by said osteoendoscopic cylinder,
at least one fastening member associated with said handle for releasable engagement of said laparoscope, and
a directional guide associated with said distal endoscopic end having a directional surface providing laminar flow of said irrigation fluid longitudinally towards a zone of visualization associated with the surgical site.

10. A laminar flow system presenting a zone of visualization at a surgical site, said system comprising:
a laminar flow endoscope including an osteoendoscopic cylinder having a laminar axis, an outer contact surface encircling an inner visual surface and extending between a proximal handle end and a distal endoscopic end;
a handle extending from the proximal handle end and presenting a lateral port of entry for coaxially receipt of a surgical instrument along the inner visual surface;
a side opening associated with said proximate handle end and in fluid communication with said distal endoscopic end; and
a directional guide associated with said distal endoscopic end having a directional surface providing laminar flow of said irrigation fluid towards the surgical site, whereby the irrigation fluid is transmitted along said laminar axis longitudinally from said distal endoscopic end through said direction guide along said laminar axis.
